(19) United States
(12) Patent Application Publication
(10) Pub. No.: US 2002/0076699 A1
(43) Pub. Date: Jun. 20, 2002

(54) Smart biochip arrays and systems and methods for using the same

(76) Inventors: Daniel Peana, Gilbert, AZ (US); Eugene Barash, Scottsdale, AZ (US)

Correspondence Address: Robin M. Silva, Esq., FLEHR HOHBACH TEST ALBRITTON & HERBERT LLP, Four Embarcadero Center, Suite 3400, San Francisco, CA, 94111, US

(21) Appl. No.: 09/738,083

(22) Filed: Dec. 16, 2000

(51) Int. Cl. C12Q 1/68; G06F 19/00; G01N 33/48; G01N 33/50; H01L 35/24

(52) U.S. Cl. 435/6; 702/20; 257/40

(57) ABSTRACT

A smart biochip array comprises an array of test sites disposed on a substrate and a smart circuit operatively associated with the substrate. Each test site contains a distinct type of probe molecule, and the smart circuit includes a memory that stores data identifying which distinct type of molecule is stored in each test site. The test sites in the biochip array are exposed to a sample that may contain target molecules capable of binding with probe molecules contained in one or more test sites. A system for scanning the exposed smart biochip array includes a detector for detecting binding between probe molecules and target molecules in each test site and a reader for reading data from the smart circuit, including data identifying the type of probe molecules contained in each test site.

### BACKGROUND OF THE INVENTION

1. Field of the Invention

This invention relates to the field of biochip arrays. More particularly, this invention relates to a smart circuit that stores information about a biochip array and to a system and method for scanning the biochip array and reading data from the smart circuit.

2. Description of Related Art

In many applications, it is desirable to detect the presence of one or more types of target molecules in a sample. For samples of biological interest, such target molecules may include antigens, antibodies, or nucleic acids. One method for detecting target molecules in a sample involves the use of probe molecules that bind to only specific types of target molecules that may be present in the sample to form bound complexes. For example, if antigens are to be detected, antibodies specific for those antigens can be used as the probe molecules. Similarly, antigens can serve as the probe molecules to detect the presence of antibodies to that antigen. Additionally, nucleic acids can be detected by probe molecules having a sequence of nucleotides that is complementary to at least a portion of the nucleotide sequence in the nucleic acid, so that the probe molecules will hybridize with the target nucleic acids.

In order to detect target molecules, the probe molecules are disposed in a test site, and the sample is added to the test site to allow the probe molecules to bind with any target molecules present in the sample. In order to achieve more efficient testing, biochip arrays are often used. Such biochip arrays provide a plurality of test sites in a single substrate. Each test site may contain a different kind of probe molecule, and the different types of probe molecules may detect different target molecules that may be present in the sample. By using a plurality of test sites, more detailed information can be obtained about the sample with only a single exposure of the biochip array to the sample. Examples of such biochip arrays are the CodeLink™ Expression Bioarrays available from Motorola, Inc. Biochip arrays are also disclosed in U.S. Pat. No. 5,874,219.

The binding of probe molecules with target molecules can be detected in a number of different ways, such as by measuring changes in the electrical or optical characteristics of the test sites. Fluorescent detection is a commonly used method of optical detection. In this approach, the target molecules in a sample are labeled with fluorescent dyes (described herein as “fluorophores”) before they are exposed the probe molecules. Such fluorophores emit fluorescence radiation in a characteristic range of wavelengths when exposed to electromagnetic radiation at a characteristic excitation wavelength. Thus, the probe molecules will bind with target molecules to form bound complexes that are fluorescent. Binding can then be detected by illuminating the test sites with electromagnetic radiation at the excitation wavelength and sensing whether the bound complexes in the test sites emit fluorescence radiation.

Another approach for fluorescent detection uses selective dyes, which are dyes that exhibit substantial changes in their optical properties in the presence of bound complexes. For example, ethidium bromide is a fluorescent dye that exhibits an approximate 20-fold increase in fluorescence when intercalated into hybridized nucleic acids, as compared to when the dye is unbound or bound only to unhybridized nucleic acids.

Once a biochip array has been exposed to the sample, specialized instruments are often used to detect binding between the probe molecules and target molecules in each test site. An example of such an instrument is the GenePix™ scanner, which is available from Axon Instruments, Inc., Foster City, Calif. Instruments for reading exposed biochip arrays are also disclosed in U.S. Pat. Nos. 5,585,639; 5,874,219; and 5,991,030.

Although such instruments can be used to rapidly and accurately determine which test sites in an exposed biochip array contain bound complexes, a weak link in the process remains, namely, the knowledge of what type of probe molecule is contained in each test site and at what concentration. Specifically, a typical biochip array may contain a large number of test sites, sometimes as many as more than ten thousand. However, the process of manufacturing a biochip array and loading it with probe molecules may occur well in advance of the time that it is exposed, and it may be performed by different persons in different locations as then expose the biochip array to the sample. Accordingly, a significant potential exists that the crucial information of which type of probe molecule has been placed in which test sites in a given biochip array, and at what concentrations, may become lost or may not be transmitted reliably. If this occurs, then the results of any tests using the biochip array cannot be properly interpreted. Because biochip arrays may be used for medical testing, such incorrect or inadequate information may be life threatening.

Accordingly, a need exists to provide a more reliable way of maintaining and transmitting crucial information regarding the probe molecules places in the test sites of biochip arrays.

### SUMMARY OF THE INVENTION

In a first principal aspect, the present invention provides a smart biochip array. The smart biochip array comprises substrate, a plurality of test sites disposed in the substrate, and a smart circuit operatively associated with the substrate. Each one of the plurality of test sites contains probe molecules of a predetermined type. The smart circuit includes a memory that stores data identifying said predetermined type of probe molecules contained in each one of the plurality of test sites.

In a second principal aspect, the present invention provides an improvement to a system for scanning an exposed biochip array. The exposed biochip array comprises a substrate having a plurality of test sites disposed therein. Each one of the plurality of test sites contains probe molecules of a predetermined type and sample molecules. The system includes a detector for detecting binding between the probe molecules and the sample molecules in each one of the plurality of test sites. The improvement comprises a reader for reading data from a smart circuit. The data identifies the predetermined type of probe molecules contained in each one of the plurality of test sites.

In a third principal aspect, the present invention provides a method for using a biochip array. The biochip array comprises a substrate having a plurality of test site disposed therein. Each one of the plurality of test sites contains probe molecules of a predetermined type, capable of binding with target molecules that may be present in a sample. The method comprises the steps of: (1) exposing the plurality of test sites to a sample; (2) scanning the plurality of test sites to detect binding between the probe molecules and the target molecules; and (3) reading data from a smart circuit, the data identifying the predetermined type of probe molecules contained in each one of the plurality of test sites.

### BRIEF DESCRIPTION OF THE DRAWINGS

FIG. 1 is a top schematic view of a smart biochip array, in accordance with a preferred embodiment of the present invention.

FIG. 2 is a block diagram of the smart circuit of FIG. 1, in accordance with a preferred embodiment of the present invention.

FIG. 3 is a block diagram of a scanning system for the smart biochip array of FIG. 1, in accordance with a preferred embodiment of the present invention.

### DETAILED DESCRIPTION OF THE INVENTION

FIG. 1 shows a top schematic view of a smart biochip array 10 in accordance with a preferred embodiment of the present invention. Smart biochip array 10 comprises a substrate 12 in which are disposed test sites 14-20. Although FIG. 1 shows four test sites 14-20, in general, array 10 may have a greater or fewer number of test sites. Each of test sites 14-20 contains a distinct type of probe molecule that is capable of binding with target molecules suspected of being present in a sample. More particularly, the probe molecules in test sites 14-20 are typically chosen to bind to only specific types of target molecules of biological interest. Such target molecules may include antigens, antibodies, or nucleic acids. Thus, if antigens are the probe molecules to be detected, then antibodies specific for those antigens can be used as the probe molecules in test sites 14-20. Similarly, antigens can serve as the probe molecules used to detect the presence in the sample of antibodies to that antigen. Additionally, nucleic acids can be detected by probe molecules having a sequence of nucleotides that is complementary to at least a portion of the nucleotide sequence in the nucleic acid, so that the probe molecules will hybridize with the target nucleic acids. Binding in test sites 14-20 may be detected optically, electrically, or by other means, using additional apparatus, such as the exemplary apparatus described in more detail below.

Test sites 14-20 may be provided in substrate 12 in various ways. For example, in some embodiments substrate 12 may be a glass slide, in which case test sites 14-20 may be defined by chambers formed into the glass slide. In other embodiments, substrate 12 may be a glass slide at least partially covered by a layer of gel, in which case test sites 14-20 may be defined by regions in the gel that contain the probe molecules. Still other ways of providing test sites 14-20 in substrate 12 may be used.

As shown in FIG. 1, a smart circuit 22 is preferably attached to substrate 12. More particularly, smart circuit 22 may be permanently affixed to substrate 12, or smart circuit 22 may be temporarily attached to substrate 12. Alternatively, smart circuit 22 may be provided in a structure, such as a plastic card, that is operatively associated with, yet physically separate from, substrate 12.

As described in more detail below, smart circuit 22 is preferably a circuit of the type used in what are typically referred to as “smart cards” or “integrated circuit cards,” such as the M-Smart™ “Mercury,” “Venus,” and “Jupiter” platforms available from Motorola, Inc. Additionally, the ISO 7816 and ISO 14443 standards of the International Organization for Standardization, which standards are fully incorporated herein by reference, specify the structure and operation of standardized integrated circuit cards.

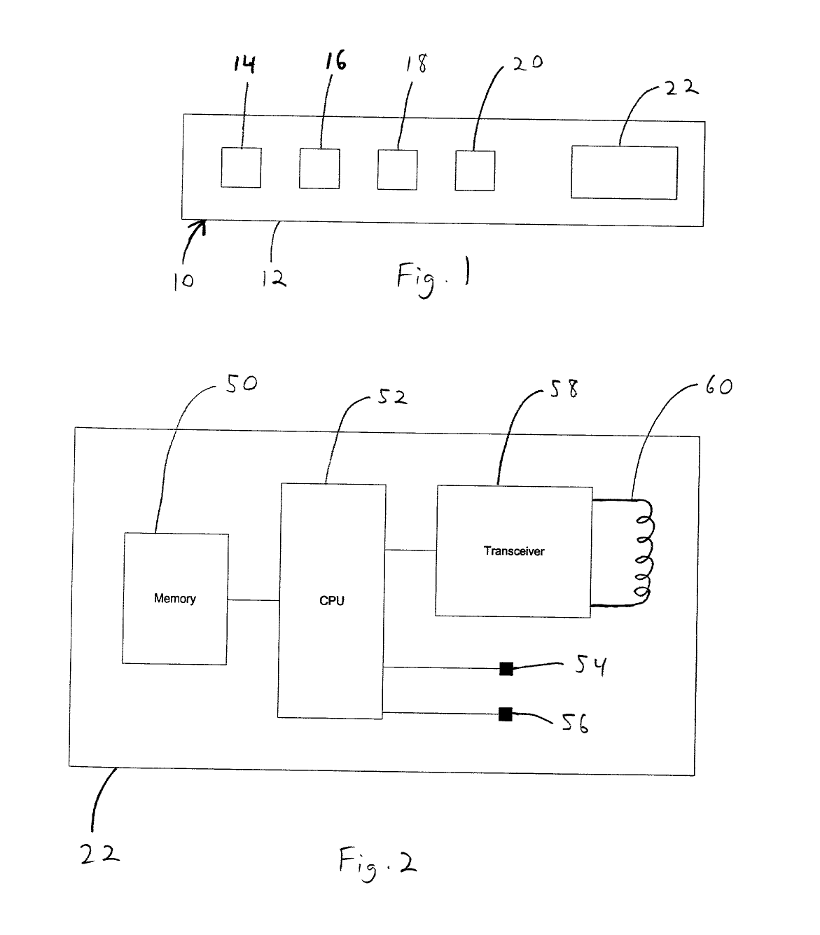

A representative embodiment of smart circuit 22 is shown in more detail in FIG. 2. Smart circuit 22 includes a memory 50, which, as described in more detail below, stores data relating to biochip array 10. Smart circuit 22 also includes components to enable a smart circuit reader/writer, described in more detail below, to read the data stored in memory 50 and to write data to memory 50. Memory 50 may include a read only memory (ROM), a random access memory (RAM), and/or an electronically erasable programmable memory (EEPROM). In preferred embodiments, smart circuit 22 includes a central processing unit (CPU) 52 coupled to memory 50 to facilitate the transfer of data between memory 50 and the smart circuit reader/writer. CPU 52 typically includes a microprocessor. In other embodiments, however, smart circuit 22 may not include a CPU, so that the smart circuit reader/writer reads data from or writes data to memory 50 directly.

Data transfer between the smart circuit reader/writer and smart circuit 22 may occur via direct electrical contact, or it may occur via a contactless interface. To allow data transfer by direct electrical contact, smart circuit 22 may include a plurality of electrical contacts, such as electrical contacts 54 and 56 coupled to CPU 52, as shown in FIG. 2. In this way, corresponding electrical contacts in the smart circuit reader/writer may make a direct electrical connection with contacts 54 and 56 for data transfer. An example of a smart circuit that uses electrical contacts for data transfer is shown and described in U.S. Pat. No. 4,841,133, which patent is fully incorporated herein by reference. A preferred direct contact interface is described in the ISO 7816 standard, though other types of direct contact interface may be used.

Preferably, however, smart circuit 22 is provided with a contactless interface for data transfer. Such a contactless interface may include a transceiver 58, which is coupled to CPU 52, and a coil 60, coupled to transceiver 58. Using this contactless interface, smart circuit 22 can send data to or receive data from the smart circuit reader/writer when coil 60 is inductively coupled to a corresponding coil in the reader/writer. An example of a smart circuit that uses a contactless interface with a coil for data transfer is shown and described in U.S. Pat. No. 5,418,353, which patent is fully incorporated by reference. A preferred contactless interface is described in the ISO 14443 standard. However, other types of contactless interfaces, such as optical interfaces, may be used. Additionally, in some embodiments, apparatus used to detect binding in test sites 14-20 may also be used as a smart circuit reader/writer to send data to or receive data from smart circuit 22.

Whether smart circuit 22 uses a direct contact or a contactless interface, smart circuit 22 is preferably highly miniaturized. For example, smart circuit 22 may occupy only one square centimeter. Moreover, smart circuit 22 is preferably substantially embedded in a fluid-impervious material, such as plastic, so that fluids used for test sites 14-20 do not damage smart circuit 22.

Memory 50 stores various data relating to smart biochip array 10. Typically, the data includes, at a minimum, the identity of the probe molecules contained in each test site 14-20. For example, each test site 14-20 may contain an oligonucleotide with a different nucleotide sequence, in which case the data stored in memory 50 would identify which nucleotide sequence corresponded to which one of test sites 14-20. Memory 50 may also store other data relating to smart biochip array 10, such as the concentrations of probe molecules contained in each test site 14-20, the date when the biochip array was charged with probe molecules, the expiration date of the biochip array, and the source of the sample to which smart biochip array 10 was exposed. More generally, memory 50 may store process information for biochip array 10, such as manufacturing data, process variables, time information, history of use information, or traffic information. The data in memory 50 may also include instructions for using smart biochip array 10. The instructions for use may, for example, specify what types of target molecules can be detected using smart biochip array 10 and what signal levels are needed to characterize a result. The data may also include information required by regulatory agencies, such as the FDA. In addition, the data stored in memory 50 may be encrypted.

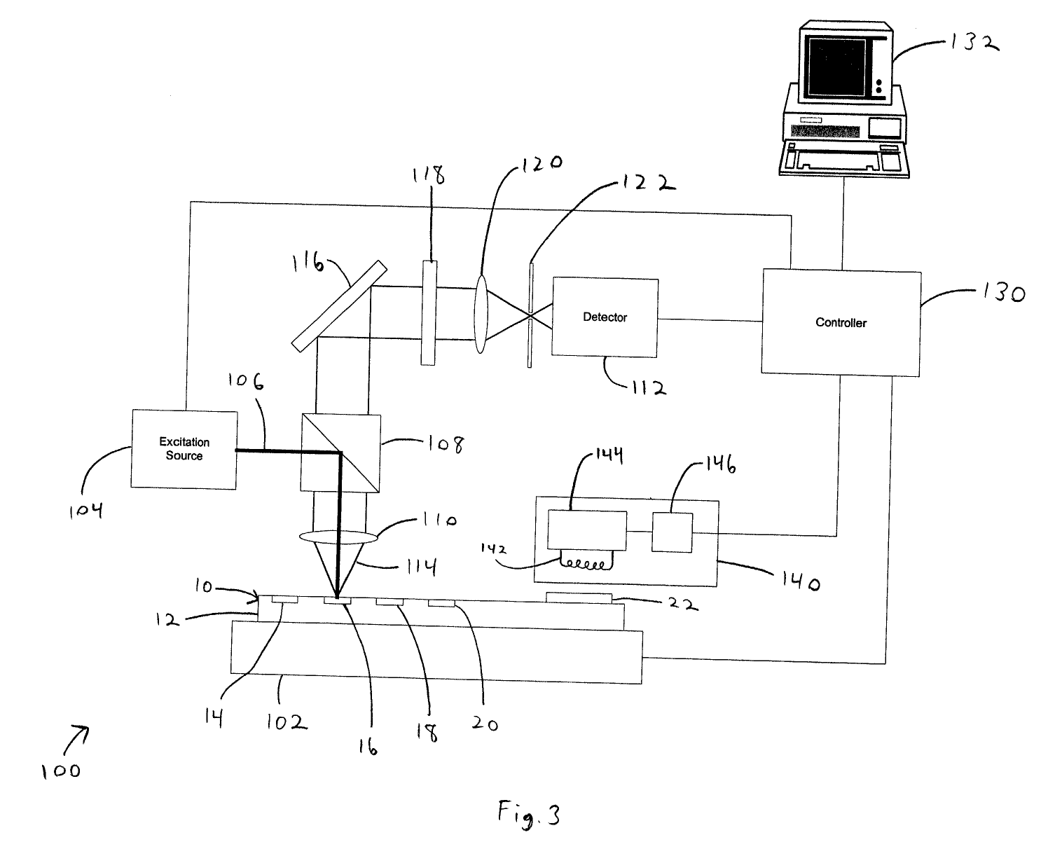

FIG. 3 shows a system 100 for reading smart biochip array 10 once it has been exposed to a sample. More particularly, system 100 shown in FIG. 3 uses fluorescent detection to detect binding between probe molecules and target molecules in the sample. However, other detection methods may also be used in system 100.

Substrate 12 of smart biochip array 10 is typically supported on a translation stage 102 in system 100, as shown in FIG. 3. Translation stage 102 allows movement in one or more directions, such as the x and y directions, i.e., movement in a plane parallel to that plane defined by substrate 12 to position test sites 14-20 appropriately in system 100.

To induce fluorescence, system 100 includes an excitation source 104 that provides excitation radiation 106, at the appropriate wavelengths, which system 100 directs to one or more of test sites 14-20 in smart biochip array 10. Preferably, excitation source 104 uses a laser to provide excitation radiation 106. Alternatively, excitation source 104 may use a broadband source, such as an arc lamp, with one or more filters to provide excitation radiation 106.

System 100 may include a source optical system to direct excitation radiation 106 to selected one or more test sites 14-20. For example, as shown in FIG. 3, excitation radiation 106 may pass through a beam splitter 108 and a lens 110 to selectively excite test site 16. However, in other embodiments, other types, configurations, and combinations of optical devices may be used to direct excitation radiation 106. For example, in other embodiments, excitation radiation 106 may be directed into more than one of test sites 14-20.

As shown in FIG. 3, system 100 also includes a detector 112 for detecting fluorescence radiation 114 emitted from smart biochip array 10. Typically, fluorescence radiation 114 will be emitted from bound complexes present in one or more of test sites 14-20 and will have been induced by excitation radiation 106. Detector 112 includes an optical sensor, such as a photo multiplier tube (PMT), photodiode, or charge-coupled device (CCD) that detects the amount of fluorescence radiation 114 admitted to it.

System 100 may also include a detector optical system for directing fluorescence radiation 114 to detector 112. For example, in the embodiment shown in FIG. 3, lens 110 collects fluorescence radiation 114 from test site 16 and directs it to a mirror 116, via a beam splitter 108. Fluorescence radiation 114 is reflected from mirror 116 and passes through an optical filter 118, which selectively transmits wavelengths corresponding to fluorescence radiation 114. A detector lens 120 gathers fluorescence radiation 114 transmitted through filter 118 and focuses it onto detector 112 through a confocal pinhole 122. It is to be understood that the optical devices for directing fluorescence radiation 114 to detector 112 shown in FIG. 3 are representative only, and that many other types, configurations, and combinations of optical devices could be used. For example, in other embodiments, detector 112 may detect fluorescence radiation emitted from more than one of test sites 14-20.

Typically, system 100 includes a controller 130 to automate its operation. Controller 130 receives an output of detector 112, which is typically a signal that indicates the amount of fluorescence radiation 114 from one or more of test sites 14-20 detected by detector 112. Controller 130 may also be coupled to translation stage 102 to control the position of smart biochip array 10 with respect to excitation radiation 106. In this way, controller 130 may control which one of test sites 14-20 is being scanned to detect bound complexes. Alternatively, or in addition to, controlling translation stage 102, controller 130 may control the position of excitation radiation 106 on smart biochip array 10, such as by controlling excitation source 104 or the source optical system used to direct excitation radiation 106 to array 10. Finally, controller 130 may control detector 112 and/or the detector optical system, in order to resolve specific ones of test sites 1420. In this way, controller 130 takes measurements on test sites 14-20, either individually or otherwise, that serve to determine whether binding with target molecules in the sample has taken place.

Controller 130 may be interfaced with a computer 132. Typically, computer 132 provides a user interface and is programmed with appropriate software to control the operation of system 100 through controller 130 and to store, record, and visualize the results. Alternatively, computer 132 may control system 100 directly, without the need for controller 130.

In exemplary embodiments of the present invention, system 100 also includes a smart circuit reader/writer 140, as shown in FIG. 3. Smart circuit reader/writer 140 reads data relating to smart biochip array 10 from smart circuit 22 and transmits it to computer 132 via controller 130. Smart circuit 22 may be disposed on substrate 12, as shown in FIG. 3. Alternatively, smart circuit 22 may be provided in a structure, such as a smart card, that is physically separate from substrate 12.

Preferably, smart circuit reader/writer 140 uses a contactless interface to read data from smart circuit 22. Accordingly, smart circuit reader/writer 140 may include a coil 142 for inductively coupling with coil 60 in smart circuit 22. Coil 142 is coupled to a wireless transceiver 144. Transceiver 144 is coupled to a CPU 146, which is coupled to controller 130.

Reader/writer 140 may, for example, be an M-Smart™ reader available from Motorola, Inc. However, other readers could be used. For example, the reader may use a different type of interface, such as a direct contact interface or an optical interface. Moreover, in some embodiments, some of the components of system 100 that detect binding in test sites 14-20 may also be used to read data from smart circuit 22. Thus, for example, detector 112 may be used to read data from smart circuit 22 via an optical interface.

By using smart circuit reader/writer 140, computer 132 obtains the information, from memory 50 in smart circuit 22, needed to interpret the results obtained by scanning test sites 14-20. In particular, computer 132 obtains the information identifying which type of probe molecules were placed in each test site 14-20. In cases where the data from memory 50 is encrypted, then computer 132 may decrypt the information, if the proper key or password is provided.

Although various embodiments of this invention have been shown and described, it is to be understood that various modifications and substitutions, as well as rearrangements and combinations of the preceding embodiments, can be made by those skilled in the art, without departing from the novel spirit and scope of this invention.

What is claimed is:

1. A smart biochip array comprising:
a substrate;
a plurality of test sites disposed in said substrate, each one of said plurality of test sites containing probe molecules of a predetermined type; and
a smart circuit operatively associated with said substrate, said smart circuit including a memory, said memory storing data identifying said predetermined type of probe molecules contained in each one of said plurality of test sites.

2. The smart biochip array of claim 1, wherein said smart circuit further includes a central processing unit (CPU) coupled to said memory.

3. The smart biochip array of claim 2, wherein said smart circuit further includes a contactless interface.

4. The smart biochip array of claim 3, wherein said contactless interface includes a wireless transceiver coupled to said CPU.

5. The smart biochip array of claim 4, wherein said contactless interface further includes a coil coupled to said wireless transceiver.

6. The smart biochip array of claim 1, wherein said smart circuit is attached to said substrate.

7. A system for scanning an exposed biochip array, said exposed biochip array comprising a substrate having a plurality of test sites disposed therein, each one of said plurality of test sites containing probe molecules of a predetermine type and sample molecules, said system including a detector for detecting binding between said probe molecules and said sample molecules in each one of said plurality of test sites, wherein the improvement comprises:
a reader for reading data from a smart circuit, said data identifying said predetermined type of probe molecules contained in each one of said plurality of test sites.

8. The system of claim 7, wherein said reader includes a contactless interface.

9. The system of claim 8, wherein said contactless interface includes a wireless transceiver.

10. The system of claim 9, wherein said contactless interface further includes a coil coupled to said wireless transceiver.

11. The system of claim 7, wherein smart circuit is attached to said exposed biochip array.

12. A method for using a biochip array, said biochip array comprising a substrate having a plurality of test sites disposed therein, each one of said plurality of test sites containing probe molecules of a predetermine type, said probe molecules being capable of binding with target molecules that may be present in a sample, said method comprising the steps of:
exposing said plurality of test sites to said sample;
scanning said plurality of test sites to detect binding between said probe molecules and said target molecules; and
reading data from a smart circuit, said data identifying said predetermined type of probe molecules contained in each one of said plurality of test sites.

13. The method of claim 12, further comprising the step of decrypting said data.
